(19) United States
(12) Patent Application Publication
(10) Pub. No.: US 2009/0171161 A1
(43) Pub. Date: Jul. 2, 2009

(54) STEERABLE ENDOSCOPIC INSTRUMENTS

(75) Inventors: Richard C. EWERS, Fullerton, CA (US); Robert A. VAUGHAN, Leander, TX (US)

Correspondence Address: LEVINE BAGADE HAN LLP, 2400 GENG ROAD, SUITE 120, PALO ALTO, CA, 94303, US

(73) Assignee: USGI Medical, Inc., San Clemente, CA (US)

(21) Appl. No.: 12/332,229

(22) Filed: Dec. 10, 2008

Related U.S. Application Data
(60) Provisional application No. 61/012,742, filed on Dec. 10, 2007.

(51) Int. Cl. A61B 1/018

(52) U.S. Cl. 600/149; 600/104

(57) ABSTRACT

Flexible, steerable, endoscopic instruments include a handle, a flexible shaft, and an end effector. The handle includes an actuator for controlling the end effector. The handle also includes a steering mechanism for steering the endoscopic instrument. The steering mechanism includes one or more tensioning members configured to increase or decrease a tension force on one or more steering wires that are attached to one or more steerable portions of the flexible shaft.

### RELATED APPLICATION DATA

This application claims priority to and the benefit of the filing date of U.S. Provisional Patent Application Ser. No. 61/012,742 (Attorney Docket No. USGIPZ07100), filed Dec. 10, 2007. The foregoing application is hereby incorporated by reference in its entirety.

### FIELD OF THE INVENTION

The present disclosure pertains to devices and methods for endoscopic procedures, including diagnostic and therapeutic procedures.

### BACKGROUND OF THE INVENTION

In endoscopic procedures, a practitioner will often use a viewing scope to intubate and view a patient. The viewing scope can be flexible or rigid. Commonly, the scope can be elongated, flexible, and have a controllable distal end. The controllable distal end typically will provide the ability to curve or steer approximately the last 2-10 cm of the viewing scope. This provides the capability to direct the scope into the lumen to be intubated or to direct the scope at points of interest. This allows the practitioner to perform visual diagnostic assessment of the patient. Scopes are commonly configured with a lumen running from the proximal to the distal end through which accessory instruments may be passed. These instruments can be electrosurgical probes, needles, graspers, biopsy cups, and other instruments known to those skilled in the art. These instruments are typically elongated and flexible. They pass through the steering section of the viewing scope and can be directed simultaneously with the scope as the scope is steered.

### SUMMARY

In one general aspect, an endoscopic instrument according to the present invention includes a mechanism for directing or steering the endoscopic instrument independently of a viewing scope. This steering mechanism provides the endoscopic instrument with the capability to be maneuvered around the visual field of the viewing scope while allowing the scope motion and instrument motion to be used independently or in combination to perform more complex endoscopic therapies. Several advantages are obtained by having an endoscopic instrument with independent steering. Some examples of these advantages are illustrated by the descriptions below.

Inserting a medical instrument through a scope 50 that is in a bent or tortuous configuration can be difficult. This is due to the fact that the instrument, a grasper 52 for example, will typically include rigid and elongated sections (the jaws, activation linkages, housing, etc.). (See, e.g., FIG. 1). Accordingly, there is usually a balance that must be incorporated between the instrument outside diameter (“OD”), the inside diameter (“ID”) of the instrument lumen contained in the viewing scope, and the size and shape of the curve that the tool will fit around. The steering sections of several embodiments of medical instruments described herein do not add any substantial additional rigid section to the instrument. This is achieved, for example, by providing a steering section 54 that is capable of bending in at least two directions. (See, e.g., FIG. 2). In other examples, the steering section 54 is bendable in four directions or even “infinite” directions (planes). (See, e.g., FIG. 3). As a result, the steering section is placed in a relaxed or passive configuration when the instrument is loaded, allowing it to take on the shape to the scope working lumen. Thus the effective “rigid length” of the tool is not increased.

End effectors 56 for several embodiments of the medical instruments described herein include a jaw or scissor structure, or the like. End effectors 56 that have these constructions typically have a plane in which the end effector is actuated. With a single plane or “back and forth” steering, the actuation plane and the steering plane are pre-determined and unalterable. (See, e.g., FIG. 4). For example, the user can only steer right or left with the jaws opening up and down. The user would not be able to steer right or left with the jaws in the same plane. If, instead, the steering capability is in four (or infinite) directions, the user can steer the tool and open the jaws in every possible configuration through combinations of steering and shaft rotation. (See, e.g., FIGS. 5-7).

The user controls for an endoscopic medical instrument tend to become more complex when the numbers of functional capabilities and degrees of freedom ate increased. For example, in several endoscopic instrument embodiments, there is provided a squeeze/release of the end effector action, an activation lock able/disable, a right/left steering wheel or lever, an up/down steering wheel or lever, and locks for freezing separately or simultaneously the steering motions. In several embodiments, advantageously, the endoscopic instrument includes a locking mechanism that is configured to automatically lock the steering mechanism at whichever position the user desires. This eliminates the need to have and activate a separate steering lock(s).

In a second general aspect, a method for performing an endoscopic procedure includes the steps of providing a steerable endoscopic device, moving the endoscopic device to a target site (e.g., tissue, organ, vessel, or other location), and pushing or pulling on a lever to steer an end effector of the endoscopic device. In several embodiments, the method includes the additional step of engaging a locking mechanism to lock the steering lever in place.

### BRIEF DESCRIPTION OF THE DRAWINGS

FIG. 1 is a schematic diagram of an endoscopic grasper located within a working lumen of an endoscope.

FIG. 2 is a schematic diagram of another endoscopic grasper located within a working lumen of an endoscope. The endoscopic grasper includes a steering section capable of bending in at least two directions.

FIG. 3 is a schematic diagram of another endoscopic grasper located within a working lumen of an endoscope. The endoscopic grasper includes a steering section capable of bending in at least four directions.

FIG. 4 is a schematic diagram of an endoscope having an endoscopic instrument extending through a working channel.

FIG. 5 is a schematic diagram of an endoscope having a steerable endoscopic instrument extending through a working channel.

FIG. 6 is a schematic diagram of an endoscope having a steerable endoscopic instrument extending through a working channel.

FIGS. 7A-C are schematic diagrams of an endoscope having a steerable endoscopic instrument extending through a working channel.

FIG. 8 is a perspective view of a flexible endoscopic instrument having a handle with an actuation mechanism and a steering mechanism.

FIG. 9 is a perspective view of the flexible endoscopic instrument shown in FIG. 8 with a portion of the handle housing removed.

FIG. 10 is a perspective view of the flexible endoscopic instrument shown in FIG. 8 with a portion of the handle housing and steering housing removed.

FIG. 11 is a close-up view of the flexible endoscopic instrument shown in FIG. 10.

FIG. 12 is a schematic view of an alternative embodiment of a flexible endoscopic instrument having a handle with an actuation mechanism and a steering mechanism.

FIG. 13 is a schematic view of a spherical tensioning member of the flexible endoscopic instrument shown in FIG. 12.

FIG. 14 is a schematic view of the spherical tensioning member shown in FIG. 13, also showing slots formed on the internal surface of the steering housing.

FIG. 15 is a schematic view of an alternative embodiment of the spherical tensioning member of the flexible endoscopic instrument shown in FIG. 12.

FIG. 16 is a perspective view of a steering section of the flexible endoscopic instrument shown in FIG. 8.

FIG. 17 is a perspective view of an end effector of the flexible endoscopic instrument shown in FIG. 8.

FIG. 18 is a schematic view of a flexible endoscopic instrument having a handle with an actuation mechanism and a steering mechanism and having a shaft that includes two separately controlled steering sections.

FIGS. 19A-D are graphic representations of alternative steering capabilities of flexible endoscopic instruments described herein.

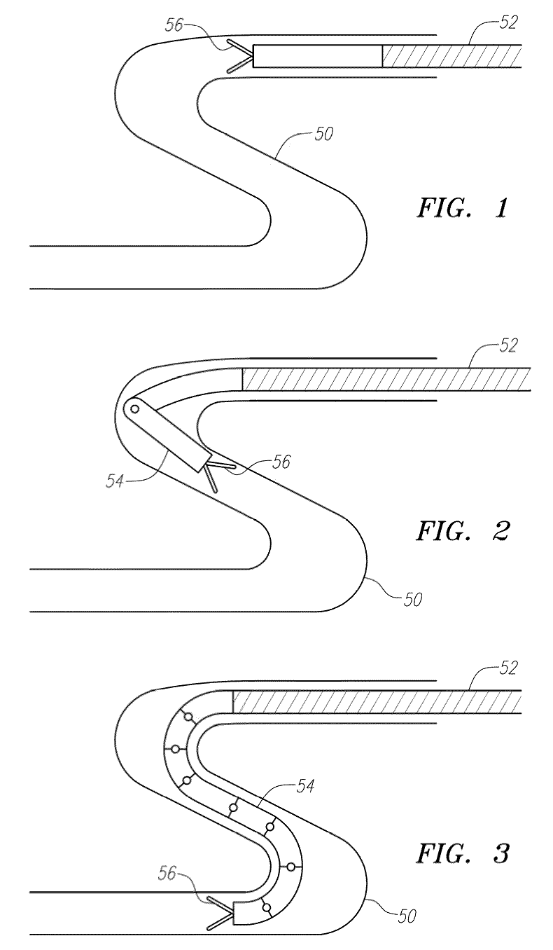

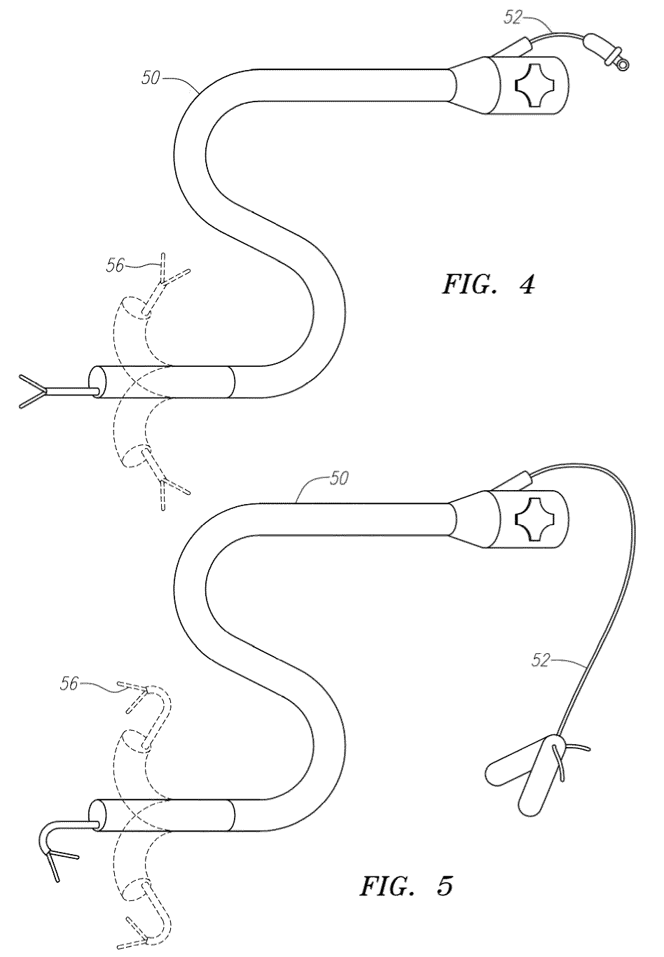

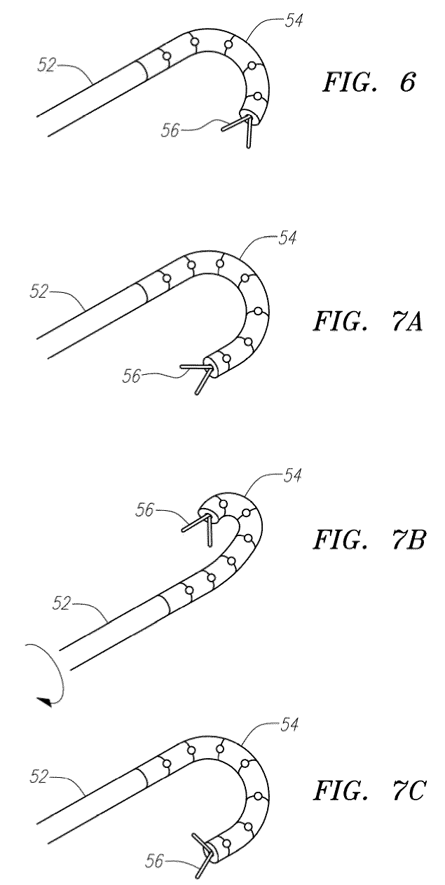

### DETAILED DESCRIPTION

Endoscopic, laparoscopic, endolumenal, and translumenal diagnostic and surgical methods and devices are described herein. In several embodiments, the methods entail performing procedures by gaining access to the internal organs of a patient through the patient's mouth or other natural orifices, reducing or eliminating the need for external incisions into the body. Operating through the body's natural orifices offers promise for faster healing times, less scarring and less pain which could lead to reduced hospitalization and quicker recovery. In other embodiments, access is gained through an access port, such as a minimally invasive access port, such as a laparoscopic access port. USGI Medical, Inc. of San Clemente, Calif. has developed several devices and methods that facilitate endoscopic, laparoscopic, endolumenal, and translumenal diagnostic and therapeutic procedures. Several endoscopic access devices are described, for example, in the following United States patent applications:

TABLE 1

| U.S. patent application Ser. No. | Filing Date |
|---|---|
| 10/346,709 | Jan. 15, 2003 |
| 10/458,060 | Jun. 9, 2003 |
| 10/797,485 | Mar. 9, 2004 |
| 11/129,513 | May 13, 2005 |
| 11/365,088 | Feb. 28, 2006 |
| 11/738,297 | Apr. 20, 2007 |
| 11/750,986 | May 18, 2007 |
| 12/061,591 | Apr. 2, 2008 |

Several tissue manipulation and tissue anchor delivery devices are described in the following United States patent applications:

TABLE 2

| U.S. patent application Ser. No. | Filing Date |
|---|---|
| 10/612,109 | Jul. 1, 2003 |
| 10/639,162 | Aug. 11, 2003 |
| 10/672,375 | Sep. 26, 2003 |
| 10/734,547 | Dec. 12, 2003 |
| 10/734,562 | Dec. 12, 2003 |
| 10/735,030 | Dec. 12, 2003 |
| 10/840,950 | May 7, 2004 |
| 10/955,245 | Sep. 29, 2004 |
| 11/070,863 | Mar. 1, 2005 |

Endoscopic tissue grasping devices are described in several of the United States patent applications listed above, and in the following United States patent applications:

TABLE 3

| U.S. patent application Ser. No. | Filing Date |
|---|---|
| 11/736,539 | Apr. 17, 2007 |
| 11/736,541 | Apr. 17, 2007 |

Tissue anchors are described in several of the United States patent applications listed above, and in the following United States patent applications:

TABLE 4

| U.S. patent application Ser. No. | Filing Date |
|---|---|
| 10/841,411 | May 7, 2004 |
| 11/404,423 | Apr. 14, 2006 |
| 11/773,933 | Jul. 5, 2007 |

Each of the foregoing patent applications is hereby incorporated by reference in its entirety.

Steerable Endoscopic Instruments

The devices described herein include several embodiments of steerable endoscopic instruments. The steerable endoscopic instruments are adapted for use during open surgery, laparoscopic surgery, endoscopic surgery, or translumenal surgery.

In a first aspect, an endoscopic surgical or diagnostic instrument is between 25 and 200 cm in length. It has an OD of 1 to 10 mm. The shaft portion of the instrument may be made rigid (or semi-rigid) in some embodiments, but it is preferably substantially flexible over all or most of its length. In a preferred configuration, the shaft is flexible from the distal tip to approximately the last 5 to 20 cm nearest the proximal end, which is semi-flexible or rigid. The distal-most 2 to 10 cm of the device can be steered by utilization of a control at the proximal handle end. In a preferred embodiment, the device can be steered in two distinct planes (e.g., up-down, right-left) which, when blended, allows infinite steering planes. Steering is accomplished via bendable materials and pull wires, pneumatics, piezoelelectric, heat controlled nitinol tendons, or other mechanisms known to those skilled in the art. In an embodiment, steering is provided through provision of a series of finite elements that are pivotably connected in an alternating pattern of 180 degrees out of phase, in a manner similar to a universal joint. The pivoted steering section is affixed to an elongated flexible, torqueable, axially translatable tube or shaft. In an embodiment, the shaft contains a plurality (e.g., four) of close-packed coils that run its entire length. Steering control wires extend through the coils from the proximal end of the shaft to the distal end. These coils serve to isolate the tension forces applied on the steering control wires to the steering section located at the distal end of the shaft. An additional (e.g., fifth) coil can optionally be provided to house the end effector activation wire, if needed. In an embodiment, the steering wires run out of the shaft/coil body and through the “universal joints” at four distinct quadrants. Tensioning of any given wire will cause the steering joint to bend toward the quadrant in which the tensioned steering wire is located. Tensioning of multiple wires will create a “blended” steering, thereby providing steering in an infinite number planes. When none of the wires is under tension, the wires will allow the steering section to be passive and externally bendable in any plane.

In another aspect, a handle includes a squeeze lever that is operably constructed to activate/deactivate the end effector. The handle and squeeze lever optionally has a spring that is biased in either of the activated or deactivated configurations. In an embodiment, the handle has the ability to lock or release the activation at any location during its movement.

In yet another aspect, the endoscopic instruments include a steering mechanism having a tensioning member configured to create a tensioning force in one or more steering wires. In some embodiments, the steering mechanism includes two wheels or levers, each attached to a drum (tensioning member). In some of these embodiments, the levers are located on the front and top of the squeeze lever, thereby being readily accessible to the user's thumb. In an embodiment, steering is accomplished through a plurality (e.g., two) levers that each move in an arc forward and backward. Moving each of the levers rotates a drum (to which each lever is attached) around an axle that is fixed to the handle. Each of the drums wraps and unwraps one or more steering wires to create and release tension. In an embodiment, each lever and drum assembly includes a tooth that engages a slot in the fixed axle (similar to a round elongated gear) that provides a position selection function. The control levers are each configured in a spring loaded yolk form. Steering is accomplished by gently pushing a lever down and then forward or backward. The pressing down releases the yolk from the axle, thereby freeing drum rotation. Pressing forward or backward then rotates the drum for steering tension. By releasing the lever from the gentle down press the yolk re-engages the slotted axle and the lever becomes locked at a discrete new position, hence locking the steering.

In other embodiments, the steering mechanism includes a tensioning member in the form of a sphere or other three-dimensional object supported in a frame and movable under control of a lever or other suitable interface. In some of these embodiments, the lever is located on the front and top of the handle squeeze lever, thereby being readily accessible to the user's thumb. In an embodiment, the lever is capable of rotating the sphere in any direction within the support frame. As the sphere rotates, it wraps and unwraps one or more steering wires to create and release tension. In an embodiment, the lever and sphere assembly includes a tooth that engages a slot in a fixed member that provides a position selection function. The control lever is configured in a spring loaded yolk form. Steering is accomplished by gently pushing the lever down and then rotating the sphere. The pressing down releases the yolk from the axle, thereby freeing sphere rotation and steering tension. By releasing the lever from the gentle down press the yolk re-engages the slot in the fixed member and the lever becomes locked at a discrete new position, hence locking the steering.

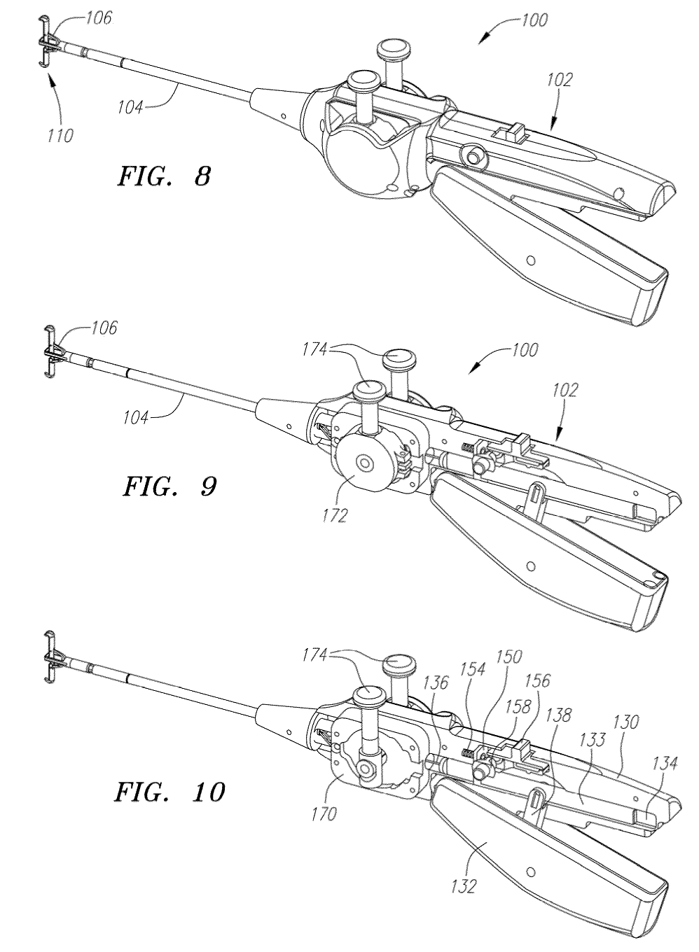

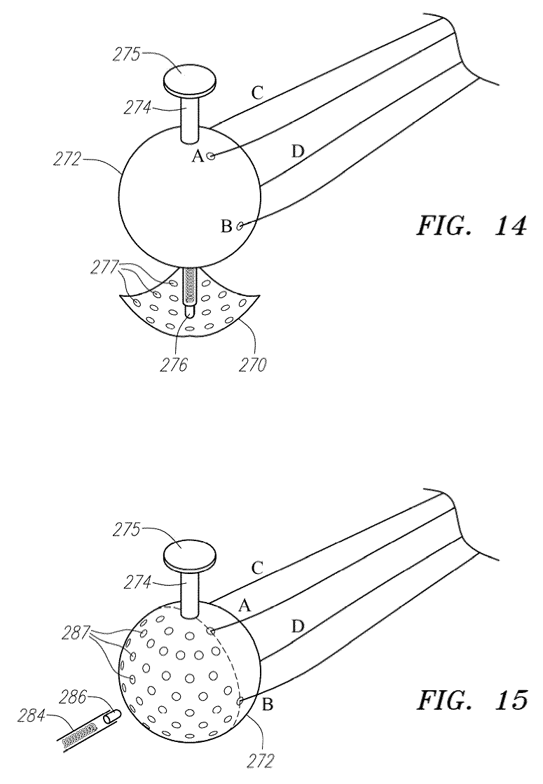

Turning to the embodiments shown in FIGS. 8 through 17, there is shown a steerable endoscopic instrument 100 having “four-way” steering—i.e., having four steering cables attached to a steering section of the shaft. As discussed above, more or fewer steering cables are included in alternative embodiments. Also, in the embodiments shown, the instrument comprises a tissue grasping tool having an end effector that includes the implements of a tissue grasper. It is contemplated that other embodiments will include end effectors having different constructions and that are capable of providing other functional capabilities, such as tissue probes, needles, graspers, biopsy cups, forceps, or the like.

The instrument includes a handle 102, a shaft 104, and an end effector 106. In the embodiment shown, the end effector 106 comprises a tissue grasper 110 having a pair of jaws 112 that are pivotably coupled to a central shaft portion 114. The jaws 112 are actuated by a pair of links 116, each of which is pivotably coupled at a distal end to one of the jaws 112, and coupled at a proximal end to an inner shaft 136 that extends through the shaft 104 of the instrument. (See FIG. 17).

The handle 102 includes an elongated main body portion 130 and a squeeze handle 132 that is pivotably attached to the main body. A sliding block 133 is retained within an elongated slot 134 in the main body 130 and is configured to move longitudinally within the slot. The sliding block 133 is attached at its proximal end to the distal end of the inner shaft 136, which extends through the shaft 104 to the end effector 106 at or near the distal end of the instrument. A linkage arm 138 is pivotably attached to the squeeze handle 132 at a first end. The linkage arm 138 includes a slot near its second end, opposite the first end. A pin attached to the sliding block 133 resides in the slot on the linkage arm, thereby allowing the linkage arm to rotate around the pin as the sliding block translates within the slot 134 in the main body 130.

In operation, as the squeeze handle 132 is “squeezed” by the user and is thereby moved toward the main body portion 130, the linkage arm 138 converts the rotational motion of the squeeze handle 132 into translational motion of the sliding block 133. Translational motion of the sliding block 133 causes the inner shaft 136 to advance or retract within the shaft 104, thereby actuating the end effector.

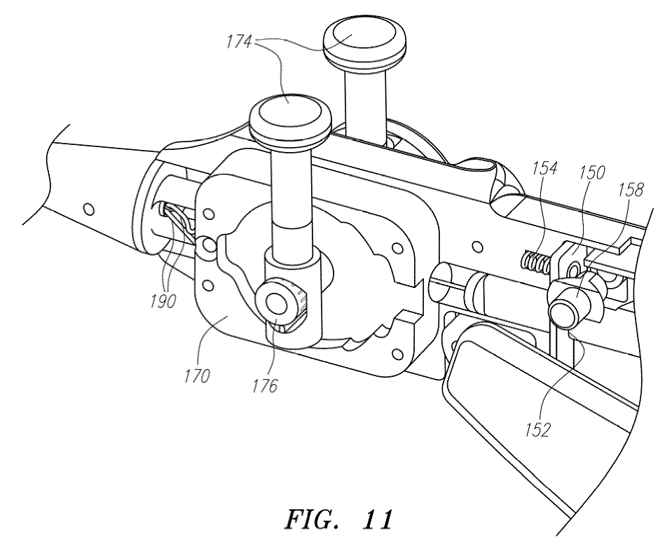

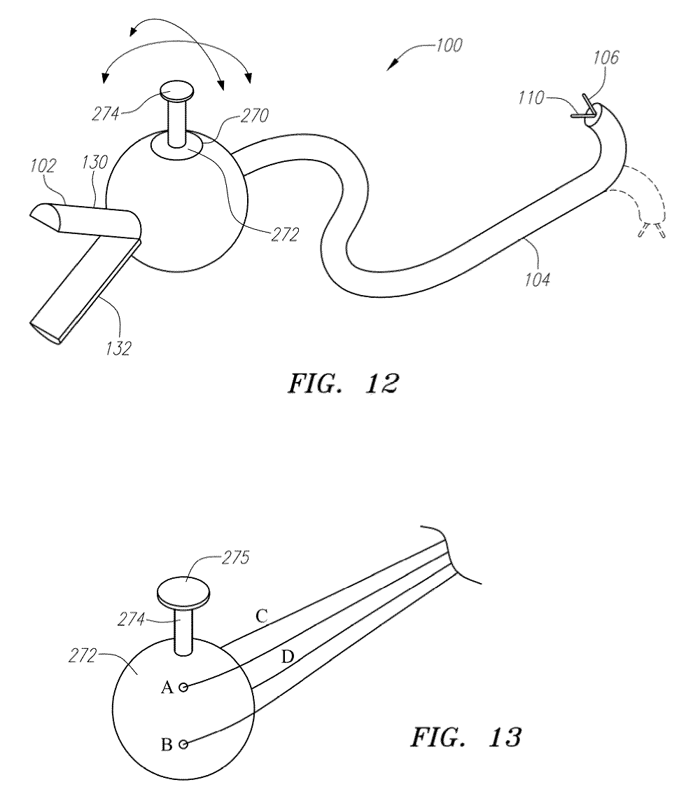

The handle embodiment shown in the figures includes a stop mechanism that is different from conventional ratchet mechanisms. A slotted plate 150 is retained within the main body portion 130 of the handle in a manner that allows the slotted plate 150 to rotate slightly in one direction. For example., in the embodiment shown, the slotted plate 150 is pivotably attached at its bottom end (e.g., behind a portion of the squeeze handle 132 in FIGS. 10 and 11), but its upper end is able to rotate slightly in the clockwise direction (and is biased in that direction by the spring 154). The slotted plate 150 is positioned substantially perpendicularly to the sliding block 133, which extends through a slot 152 formed in the slotted plate 150. The size of the slot 152 and the relative orientation of the slotted plate 150 relative to the sliding block 133 provides the slotted plate 150 with the capability of allowing the sliding block 133 to move in one direction (e.g., advancing) while preventing movement in the other direction (e.g. retraction). In the embodiment shown, there are no teeth or pawls on the components, and therefore no ratcheting mechanism is formed. A sliding switch 156 is able to be advanced into a forward position to override the operation of the slotted plate 150, thereby allowing free movement of the sliding block 133 in either direction. A release switch 158 can be pushed to disengage the slotted plate 150 from the sliding block 133 when the sliding switch 156 is not in use.

Turning to the steering mechanism, in the embodiment shown in FIGS. 8-11, a steering housing 170 is attached to (or formed integrally within) the main body portion 130 of the handle. The housing 170 includes a central lumen through which the inner shaft 136 extends. Two steering drums 172 are supported within the steering housing 170, with one drum on each side of the housing. Each drum 172 is attached to two opposed steering wires that extend through coils retained within the shaft 104 and that connect to the steering section of the instrument. Accordingly, rotation of each drum 172 controls steering of the device within a given plane. A steering lever 174 extends through a hole in each drum 172, and is supported by a central shaft 176 that is fixed to the housing 170. The central shaft 176 includes a plurality of circumferential slots, and each lever 174 includes a tab that is able to selectively mate with each of the plurality of slots on the fixed shaft 176. (See FIG. 11). Accordingly, each lever 174 can be locked into position relative to the shaft 176. Each lever 174 includes a spring (not shown) that biases the lever 174 into a locked position (i.e., into engagement with the shaft 176). The user is able to release the lever 174 from the shaft by pushing downward against the spring force. Once released, the lever 174 is able to be rotated to change the steering

In an alternative embodiment of the steering mechanism, shown in FIGS. 12-15, a steering housing 270 is attached to (or formed integrally within) the main body portion 130 of the handle. The housing 270 includes a central lumen through which the inner shaft 136 extends. A steering sphere 272 is rotatably supported within the steering housing 270. The steering sphere 272 is attached to two pairs of opposed steering wires (A, B, C, and D) that extend through coils retained within the shaft 104 and that connect to the steering section of the instrument. Accordingly, rotation of the sphere 272 controls tensioning of the steering wires and thereby controls steering of the device within a given plane. A steering lever 274 extends through a hole in the steering sphere 272, and is supported by a central shaft (not shown) or other member that is fixed to the housing 270. A first end of the steering lever 274 includes a user control 275, and a second end of the steering lever 274 includes a tab 276 that is able to selectively mate with each of a plurality of slots 277 formed on an inward facing surface of the steering housing 270. (See FIG. 14). Accordingly, the lever 274 can be locked into position relative to the steering housing 270. The lever 274 includes a spring (not shown) that biases the lever 274 into a locked position (i.e., into engagement with the slots 277 in the housing 270). The user is able to release the lever 274 from the slots 277 by pushing downward against the spring force. Once released, the lever 274 is able to be rotated to change the steering position, then re-engaged with one of the plurality of slots 277 to “lock” the steering position. In an alternative embodiment, shown in FIG. 15, a locking tab 286 is provided on a locking shaft 284 separate from the lever 274. In this embodiment, the locking tab 286 is adapted to selectively engage one of a plurality of slots 287 formed over at least a portion of the surface of the steering sphere 272. Accordingly, locking of the steering mechanism is controlled by the locking shaft 284.

The steering wire coils 190 are shown in FIG. 11 extending from the steering housing 170 distally and entering the proximal end of the shaft 104. The steering wire coils were described previously, and function to isolate forces on the steering wires (which extend through the coils) to facilitate steering. The proximal ends of each of the steering wires are attached to one of the drums 176. The distal ends of each of the steering wires are attached to the steering section of the instrument.

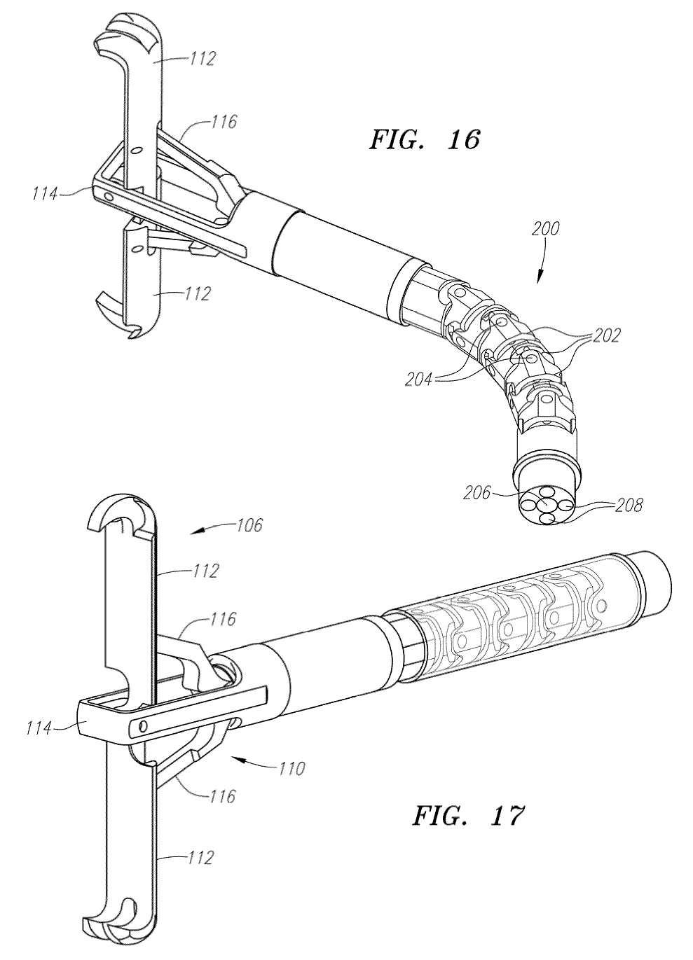

Turning to FIG. 16, a steering section 200 includes a plurality of finite links 202 located longitudinally adjacent to one another. Each link 202 is pivotably hinged to its adjacent link via a pin 204, thereby allowing adjacent links to pivot or rotate relative to one another. As noted previously, in the embodiment shown, each alternating link 202 is orientated at 180 degrees relative to its adjacent links—similar to the construction of a universal joint—thereby providing a full range of motion for the steering section 200. A central lumen 206 extends through the steering section 200 (and each of the individual links 202), providing a passage for the inner shaft 136. Four steering lumens 208 also extend through the steering section 200 (and each of the individual links 202), thereby providing a passage for each of the four steering wire coils 190 and steering wires

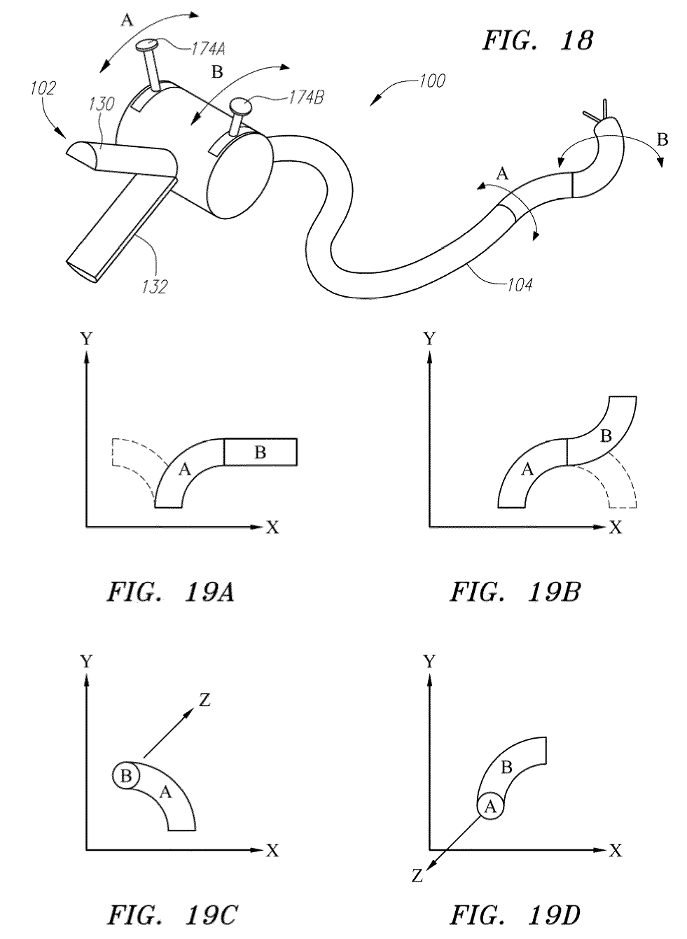

The steerable endoscopic instruments described herein include components that are adaptable to several alternative steering control configurations and combinations, as illustrated generally in FIGS. 18 and 19A-D. For example, in several of the described embodiments, each steering wire is controlled at its proximal end by a tensioning member (such as the one or more drums 172 or steering sphere 272) and is attached at its distal end to a portion of the end effector or steering section of the shaft in order to enable steering or other manipulation of the device. In this manner, two-way, four-way, or other steering capabilities are provided. In an alternative embodiment, shown in FIG. 18, a medical instrument incorporates two steering sections A and B. For example, a first steering section A is located on the shaft 104 at a location proximal to a distal steering section B. The first steering section A is controlled by a first control lever 174A, and the second steering section B is controlled by a second control lever 174B. In the embodiment shown, movement of the first steering section A is limited to a first plane, and movement of the second steering section B is limited to a second plane, with the first plane being generally perpendicular to the second plane. Other variations are also possible, such as four-way steering of each steering section, additional steering sections under control of the same or additional tensioning members, or combinations of the above.

FIGS. 19A-D illustrate the movement capabilities of two alternative two-steering section embodiments of the steerable endoscopic instruments described herein. In each of the Figures, the distal end of a steerable endoscopic instrument is represented schematically on a three-dimensional graph as having a first steerable section A and a second steerable section B. In FIG. 19A, the first steering section A is movable within the X-Y plane, as indicated by the shadowed lines, while the section steering section B is not changed. In FIG. 19B, the second steering section B is steered independently of the first steering section A to provide a compound steering capability within the X-Y plane. Similarly, in FIG. 19C, a first steering section A is movable within the X-Y plane, while the second steering section B is movable within the X-Z and/or Y-Z planes. Finally, in FIG. 19D, a first steering section A is movable within the X-Z and/or Y-Z planes, and a second steering section B is movable within the X-Y plane.

Commonly assigned U.S. patent applications Ser. Nos. 11/736,539 and 11/736,541, each of which was filed on Apr. 17, 2007, are each incorporated by reference herein in their entireties. Each of these incorporated applications describes endoscopic instruments that are suitable for incorporating the features of the instruments described in the present application. For example, the steering features and the handle mechanisms described herein are suitably substituted for the corresponding features of the devices described in the incorporated applications.

In several embodiments, the medical instruments described herein are adapted for use in engaging, grasping, and manipulating tissue during open surgery, laparoscopic surgery, endoscopic surgery, or translumenal surgery. In particular, in those embodiments, the medical instruments are adapted to engage the soft, multilayer tissue of a human or animal stomach in an endolumenal approach. Alternatively, the medical instruments may be used to engage other human or animal gastric tissue, peritoneal organs, external body surfaces, or tissue of the lung, heart, kidney, bladder, or other body tissue. In several embodiments, the instruments are useful for engaging, grasping, and manipulating tissue that is difficult to engage using conventional graspers, which frequently occurs during translumenal surgical procedures (e.g., natural orifice translumenal endoscopic surgery, or “NOTES”). Several translumenal procedures are described in U.S. patent applications Ser. No. 10/841,233, Ser. No. 10/898,683, Ser. No. 11/238,279, Ser. No. 11/102,571, Ser. No. 11/342,288, and Ser. No. 11/270,195, which are hereby incorporated by reference. The medical instruments described herein are suitable for use in combination with, for example, the endoluminal tool deployment systems described in U.S. patent application Ser. No. 10/797,485, which is hereby incorporated by reference. In particular, the tool deployment systems described in the '485 application includes one or more lumens suitable for facilitating deployment of the medical instruments described herein to perform or assist in performing endoscopic, laparoscopic, or NOTES diagnostic or therapeutic procedures. In addition, the medical instruments described herein are suitable for use in combination with, or instead of, the methods and instruments described in U.S. patent application Ser. Nos. 11/412,261, which is also incorporated by reference herein.

Two or more instruments may be used simultaneously during a procedure. For example, a medical instrument incorporating the features of the embodiments described herein may be used in combination with a conventional medical instrument. In alternative embodiments, two or more of the medical instruments described herein may be used simultaneously in a single procedure. In some embodiments, the two or more instruments are deployed to a diagnostic or surgical site using the endoluminal tool deployment systems described in the '485 application listed above.

Although various illustrative embodiments are described above, it will be evident to one skilled in the art that various changes and modifications are within the scope of the invention. It is intended in the appended claims to cover all such changes and modifications that fall within the true spirit and scope of the invention.

What is claimed is:

1. A steerable endoscopic instrument comprising:
a handle;
an elongated flexible shaft extending from the handle, the shaft comprising an outer sheath and an inner shaft member, the shaft including a steerable portion located distally of the handle;
an end effector disposed at or near a distal end of the shaft, with a distal end of the inner shaft member being operatively coupled to the end effector;
at least one steering wire extending between the handle and the steerable portion of the shaft, the at least one steering wire being operatively coupled to the steerable portion of the shaft;
an actuator associated with the handle and coupled to a proximal end of the inner shaft member such that movement of the actuator causes translation of the inner shaft member relative to the outer sheath; and
a tensioning member associated with the handle and coupled to a proximal end of the at least one steering wire such that movement of the tensioning member increases or decreases a tension force in said steering wire.

2. The instrument of claim 1, wherein said tensioning member comprises a drum rotatably supported on said handle.

3. The instrument of claim 1, wherein said tensioning member comprises a sphere rotatably supported on said handle.

4. The instrument of claim 1, further comprising a steering lock having a first position substantially preventing movement of said tensioning member and a second position allowing movement of said tensioning member.

5. The instrument of claim 4, wherein said steering lock comprises a tab and a plurality of slots.

6. The instrument of claim 5, wherein the tab is disposed on a lever used to move the tensioning member, and the plurality of slots are disposed on a portion of said handle.

7. The instrument of claim 1, wherein said steerable portion of said shaft comprises a proximal steering portion and a distal steering portion, said proximal and distal steering portions being controlled separately such that each is independently steerable.

8. The instrument of claim 1, wherein said handle comprises a pusher block slidably received within a main body, and an actuation arm connected to said pusher block by a linkage.

9. The instrument of claim 8, further comprising a stop mechanism including a plate member having a slot through which the pusher block extends, the plate member having a first position in which the plate member substantially prevents translation of the pusher block and a second position in which the plate member allows translation of the pusher block through the slot.

10. The instrument of claim 1, wherein said steerable portion of said shaft comprises a plurality of hinged links.

11. The instrument of claim 1, wherein the end effector comprises a tissue grasper having a pair of jaws.
